(19) United States
(12) Patent Application Publication
(10) Pub. No.: US 2003/0077628 A1
(43) Pub. Date: Apr. 24, 2003

(54) Method of screening compounds for biological activity

(76) Inventors: Steven William Homans, Leeds (GB); Hiroki Shimizu, Miyagi (JP)

Correspondence Address: MYERS BIGEL SIBLEY & SAJOVEC, PO BOX 37428, RALEIGH, NC, 27627, US

(21) Appl. No.: 10/204,526

(22) PCT Filed: Jan. 30, 2001
(86) PCT No.: PCT/GB01/00351

(51) Int. Cl. C12Q 1/68; G01N 33/53

(52) U.S. Cl. 435/6; 435/7.1

(57) ABSTRACT

The present invention provides a method of screening compounds to identify ligands that bind to specific target molecules using nuclear magnetic resonance (NMR) and the measurement of residual dipolar couplings. The method is particularly useful in screening and/or identifying compounds which bind to specific target molecules, for example proteins, polypeptides and macromolecules so as to assist in rational drug design.

The present invention relates to the use of nuclear magnetic resonance (NMR) to screen and/or identify compounds which bind to specific target molecules, for use especially in screening libraries of ligands and their binding to target molecules so as to assist in rational drug design.

### BACKGROUND TO THE INVENTION

The various genome sequencing projects currently underway are generating data at an enormous rate. The three-dimensional structures of the target molecules encoded by the relevant gene sequences are a suitable platform for rational drug design, i.e. the design of compounds that bind to target molecules, for example as agonists or antagonists of a natural ligand, as an inhibitor, a substrate or a target vector. For the purpose of rational drug design it is even more beneficial to have a three-dimensional structure at atomic resolution of the complex between the target molecule and the natural ligand. However, the complexity of the energetics of the binding process are currently insufficiently understood to enable rational drug design using this information alone.

It is commonplace to design a large number of compounds (a ‘library’) based upon a common chemical theme, or a reduced number of compounds (a ‘focussed library’) whose common structural skeleton is inferred from upon the three dimensional structure of the complex. These compounds are screened either individually or as mixtures in a chemical or biological assay designed to detect the desired activity (a ‘positive’). However, a problem associated with assays of this nature concerns the number of ‘false positives’ and ‘false negatives’. A false positive can arise where a member of the library binds non-specifically to the target molecule in a position other than the site of the binding (the ‘binding site’), whereas a false negative can arise where a member of the library has an affinity for the target molecule which is too low to enable detection in the assay procedure. False results can be costly for the pharmaceutical industry both in research and development time and money.

It is known from the prior art to use protein crystallography to determine the three-dimensional structures of target molecules and their complexes with ligands. However, the problem associated with this method is that a crystal of the target molecule-ligand complex is required for every member of the library, and the growth of such crystals is largely trial-and-error even for one skilled in the art. Thus it will be apparent that crystallography does not represent a method suitable for rapid screening.

Another technique that has been tried is NMR. NMR requires materials to be in solution and in principle, more than one member of a given library can be screened simultaneously. It is known from the prior art, as disclosed in U.S. Pat. Nos. 5,698,401, 5,804,390 and U.S. Pat. No. 5,891,643 to use NMR to screen libraries of putative ligands so as to identify the compound or compounds that bind to the target molecule. Each of the above techniques is based on generating a first two dimensional ¹⁵N/¹H NMR correlation spectrum from an isotopically enriched protein and a second ¹⁵N/¹H NMR correlation spectrum from the isotopically enriched protein/ligand complex. The protein spectrum changes are then used to identify the binding site. In other words the prior art technique can only give information as to the location of the binding site on the protein and whether a ligand has actually bound to the protein. Moreover the technique is restricted to isotopically enriching the protein with ¹⁵N.

The problem associated with the prior art NMR technique is that it is not possible to gain information as to orientation of members of the ligand family being screened. The prior art techniques can neither give information as to the relative orientation of the ligand family members i.e. the technique is not capable of comparative identification of the best candidate(s) from a library/set, nor is the technique able to give information as to the absolute orientation of the ligand with respect to the protein.

The present invention mitigates or overcomes these difficulties by providing a method which (a) enables the detection of a member or members of a library whose affinity or affinities are too weak to detect by conventional assays, and (b) allows discrimination between two or more members of a given library that bind with the same or different relative orientations with respect to the target molecule.

We have used a completely different approach to the problem of NMR screens for ligands based on a chemical shift approach. We have used a method based on the energy of interaction between two magnetic moments in an applied magnetic field which is dependent upon the distance between the moments and the angle formed between the vector joining the moments and the magnetic field. In the NMR spectrum of atomic nuclei that possess such moments, this energy of interaction is manifest as a ‘splitting’ of the resonance line corresponding to each nucleus. The magnitude of this splitting measured in Hertz is known as the dipolar coupling constant. The dipolar coupling constant is not observed for atomic nuclei in molecules that tumble rapidly in solution and have no net orientation (‘isotropic’ tumbling), since the average value of the angular term averages to zero. Conversely, large dipolar couplings (typically kilohertz) are observed between atoms in molecules that are rigidly aligned with respect to the applied field, i.e. the solid state.

In the present invention we describe the use of residual dipolar coupling (1) which has particular advantage in identifying weakly oriented target molecules and their complexes (2). A ligand bound to a target molecule will adopt the same degree of alignment as the target molecule, and will possess the same orientation with respect to the applied magnetic field. This contrasts with the free ligand in solution, which will possess a much smaller degree of alignment by virtue of its much smaller size.

We have used our unexpected observations to overcome the problems associated with the prior art so as to advantageously improve the sensitivity of a ligand screen, and to provide immediate information on the disposition of ‘positives’ with respect to the target molecule, thus enabling the detection of ‘positives’ that bind in the correct binding site. The present invention allows simultaneous data to be generated regarding both the ligand binding affinity and its orientation with respect to the target molecule. We believe the method of screening ligands of the present invention will be of particular use to the pharmaceutical industry.

### STATEMENT OF THE INVENTION

In its broadest aspect, the invention provides a method of screening compounds to identify ligands that bind to specific target molecules using the measurement of residual dipolar couplings.

According to a first aspect of the invention there is provided a method of identifying a ligand or ligands that bind to a specific target molecule comprising the steps of:

(i) placing at least one ligand in a liquid crystalline solution;;

(ii) generating a first one-, two- or multidimensional high resolution NMR correlation spectrum of said at least one ligand. so as to observe one- two- or multiple bond scalar couplings;

(iii) adding a sample of the specific target molecule to the at least one ligand in solution;

(iv) generating a second one-, two- or multidimensional high resolution NMR correlation spectrum of the at least one ligand and;

(v) comparing said first and second high resolution NMR correlation spectra so as to identify differences in splitting of resonance lines assigned to particular pairs of nuclei within the at least one ligand.

Preferably, the first and/or second high resolution NMR correlation spectra relate to chemical shifts of NMR active nuclei of any element which occurs in the specific target molecule.

Preferably, the second high resolution NMR correlation spectrum of the ligand is obtained under identical conditions as those for obtaining the first of said spectra so as to ensure accurate comparisons between the two can be made.

Preferably, the specific target molecule is a protein or polypeptide, optionally the target molecule may be a membrane protein in, for example, a detergent solution.

Thus it will be appreciated that the invention provides a one-, two- or multidimensional high resolution NMR correlation spectrum of the ‘natural ligand’, ligand library or selected members thereof, the ligand being provided in any dilute liquid crystalline medium. The high resolution NMR correlation spectrum is obtained in a manner that permits the observation of one- two- or multiple bond scalar couplings. The spectrum will typically correlate the chemical shifts of NMR active nuclei such as ¹H, ¹³C, ¹⁵N or ³¹P, but is not restricted to these nuclei and may be correlated to any other element of the specific target molecule. The method of the present invention is applicable to any target macromolecule.

The composition of the liquid crystalline medium is well known to those skilled in the art, and is not intended to limit the scope of the application. Nonetheless, suitable examples include any one of the following:

(i) dimyristoyl phosphatidylcholine:dihexanoylphosphatidylcholine, preferably at a concentration of 2.9:1 (mol/mol) in aqueous solution (5-15% w/v);

(ii) ditridecylphosphatidylcholine:dihexylphosphatidylcholine, preferably at a concentration of 3.0:1 (mol/mol) in aqueous solution (5-15% w/v) or;

(iii) aqueous solution of cetylpyridinium chloride:hexanol, preferably at a concentration of 1-5% (w/w) 1:1 (w/w) in 0.2 M NaCl.

Once a sample of the specific target molecule/macromolecule is added to the ligand in the dilute liquid crystalline medium, a second correlation spectrum is acquired under conditions that are otherwise identical with the first. The differences in splittings of the resonance lines are assigned to particular pairs of nuclei within the ligand or ligands, by conventional methods. Ligand library members that are ‘positives’ are identified by changes in the splittings of their resonance lines, and ‘positives’ that bind in the same binding site and with the same relative disposition arc identified by splittings that change in the same ratio when compared over all nuclear pairs.

The present invention makes use of the molecule existing in a state intermediate between the fully aligned and isotropic case, i.e. partially aligned. This latter state is induced by dissolving the molecule in any liquid-crystalline medium that imparts a small net degree of order on the molecule. In this way the residual dipolar couplings are scaled relative to their maximum values, and give rise to splittings on the order of tens of hertz. The scaling of the splittings considerably simplifies spectral interpretation, a task which is practically impossible for more than a dozen nuclei in the fully aligned state.

The use of residual dipolar couplings derives from the realisation that a ligand that is bound to a target molecule will adopt the same degree of alignment as the target molecule, and will possess the same orientation with respect to the applied magnetic field. This contrasts with the free ligand in solution, which will possess a much smaller degree of alignment by virtue of its much smaller size. Moreover, members of a library that bind to the target molecule in the same binding site and in a similar manner to the ‘natural ligand’ will exhibit the same or very similar residual dipolar couplings. The binding phenomenon is manifest in a change in the size of the splitting of the resonance lines of atoms within the ‘natural ligand’ or of members of the library that possess activity. Typically the resonance line is also split by the scalar spin-spin coupling interaction. Since the size of this coupling is constant and does not depend on alignment, the residual dipolar coupling can be measured as the difference between the size of the scalar splitting in the absence of alignment compared with its value in the partially aligned state.

In one embodiment of the invention, the method further includes the step of isotopically enriching both the ligand or a ligand library and the specific target molecule, or alternatively the ligand or a ligand library alone, with an NMR active stable isotope prior to generating the high resolution NMR correlation spectra. Such a step offers the further advantage of improving the sensitivity by virtue of the increased number of stable isotopic nuclei per unit volume of the sample. However, it will also be appreciated that this additional step is not required in order for comparable high resolution spectra to be produced, it merely offers a method of further increasing sensitivity.

In the instance of isotopically enriching the specific target molecule, either alone or with the ligand or ligand library, there is a yet further advantage to the invention in that parameters relevant to the extent and degree of alignment of the target molecule (the components of the alignment tensor) can be extracted by conventional procedures, and used to assist in the construction of high-resolution three-dimensional structures of target molecule-ligand complexes.

Preferably, the enriching NMR active stable isotope is selected from the group consisting of: ¹³C, ¹⁵N, ³¹P or ²H, or a mixture of such isotopes or radioactive isotopes thereof in any combination, or any other NMR active stable isotope or unstable isotope thereof which occurs in the ligand.

In another embodiment of the invention, the target molecule is biochemically derivatised such that it is bound strongly to the chemical species that comprise the matrix of the liquid-crystalline medium, or possesses the inherent capacity to do so. It is recognised that certain proteins may inherently contain suitable derivatives, for example membrane proteins. The derivitisation can take many forms and it is not intended to limit the scope of the application. Nonetheless, suitable examples include any one of the following:

(i) myristoylation at one or more positions on the protein;

(ii) presence of a membrane spanning domain or glycosyl-phosphatidylinositol membrane anchor either inherently by genetic engineering of an over-expressed protein or;

(iii) covalent attachment of the protein to functional groups on the liquid crystalline medium by chemical means.

This embodiment offers the further advantage that the target molecule will adopt a high degree of alignment, such that the resonance lines of ligands which bind only weakly to the target molecule (dissociation constants>10⁻⁶ molar) will show significant splitting due to residual dipolar couplings.

According to a second aspect of the invention there is provided the method of the first aspect of the invention for use in screening a library of ligands so as to select a candidate therapeutic comprising a ligand or ligands with appropriate biological activity.

Preferably, the method further includes mixing the selected ligand or ligands identified as a candiadate therapeutic, or derivative or homologue thereof with a pharmaceutically acceptable carrier.

Preferably, the method further includes any one or more of the preferred features herein before described.

According to a third aspect of the invention there is provided a method for the production of a pharmaceutical composition comprising identifying an agent ligand or ligands by the method as herein described, and furthermore mixing the agent identified, or derivative or homologue thereof with a pharmaceutically acceptable carrier.

### BRIEF DESCRIPTION OF THE DRAWINGS

The invention will now be described by way of example only, with reference to the accompanying Figures wherein:

FIG. 1 illustrates ¹³C-¹H Heteronuclear Single Quantum Correlation (HSQC) spectrum of a mixture of lactose (Galβ1-4Glc) and globotriaosylceramide oligosaccharide (Galα1-4Galβ1-4Glc), in the absence (bold lines) and presence (faint lines) of the receptor B-subunit derived from the Escherichia coli O157 toxin. Only the resonances of Galα1-4Galβ1-4Glc, the natural ligand, are shifted in the presence of the receptor. The resonances of Galβ1-4Glc, which is not a ligand for the protein. are unchanged.

### DETAILED DESCRIPTION OF THE EMBODIMENTS

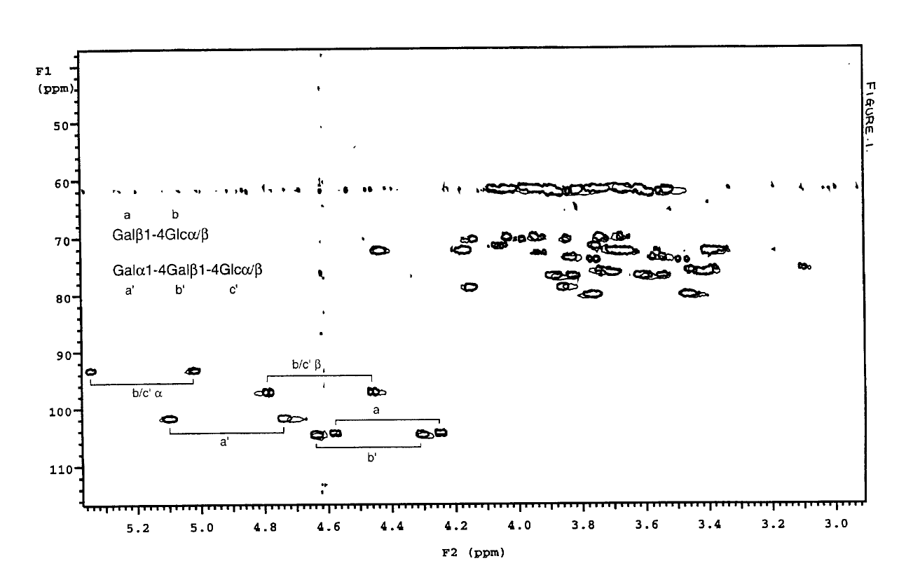

To a pre-weighed pre-washed glass septum vial (Pierce No. 13804) was added 840 ml dihexanoylphosphatidylcholine (DHPC) in chloroform solution (Sigma P4148) by use of a 250 μl Hamilton microsyringe. Chloroform was evaporated in a stream of dry nitrogen gas for 15 minutes, followed by lyophilisation for at least two hours. The vial was re-weighed to determine the exact amount of DHPC (22 mg). To the dried DHPC was added 785 μl deuterium oxide (Aldrich), followed by 95.8 mg dimyristoylphosphatidylcholine (DMPC, Sigma P6392), to give a 15% solution of DHPC:DMPC 1:2.9 (mol/mol). Once dissolution was complete, a further 785 μl deuterium oxide was added to give a 7.5% solution. To 650 μl of this solution was added uniformly ¹³C-enriched globotriaosylceramide oligosaccharide and ¹³C-enriched lactose, prepared as described (3) to final concentrations of 0.28 mM and 0.14 mM respectively. A ¹³C-¹H HSQC spectrum was recorded on this solution at 308 K and pH 7.0 without broadband ¹³C decoupling in the F2 dimension. in order that the one-bond ¹³C-¹H splittings could be observed. The relevant resonances are shown in boldface in FIG. 1. A total of 2.7 mg. lyophilised E. coli O157 B subunit receptor was then dissolved in the above solution, and a second HSQC spectrum was recorded under otherwise identical conditions to the first. The relevant resonances are shown in normal face in FIG. 1.

### REFERENCES

1. Tjanda N and Bax A, Science, 278, 1111-1114 (1997).

2. Shimizu H, Donohue-Rolfe A and Homans S W, J. Am. Chem. Soc., 121, 5815-5816 (1999).

3. Shimizu H, Brown J M, Homans S W and Field R A, Tetrahedron, 54, 9489-9506, (1998).

1. A method of identifying a ligand or ligands that bind to a specific target molecule comprising the steps of:
(i) placing at least one ligand in a liquid crystalline solution so that it is partially aligned;
(ii) generating a first one-, two- or multidimensional high resolution NMR correlation spectrum of said at least one ligand, so as to observe one- two- or multiple bond residual dipolar couplings;
(iii) adding a sample of the specific target molecule to the at least one ligand in solution;
(iv) generating a second one-, two- or multidimensional high resolution NMR correlation spectrum of the at least one ligand and;
(v) comparing said first and second high resolution NMR correlation spectra so as to identify differences in splitting of resonance lines assigned to particular pairs of nuclei within the at least one ligand.

2. A method according to claim 1 wherein the first and/or second high resolution NMR correlation spectra correlates to chemical shifts of NMR active nuclei of any element which occurs in the specific target molecule.

3. A method according to either claim 1 or 2 wherein the second high resolution NMR correlation spectrum of the ligand is obtained under identical conditions as those for obtaining the first of said spectra so as to ensure accurate comparisons between the two.

4. A method according to any preceding claim wherein the specific target molecule is a protein or polypeptide or macromolecule.

5. A method according to any preceding claim wherein the specific target molecule is a membrane protein.

6. A method according to 5 wherein the protein is provided in a detergent solution.

7. A method according to any preceding claim wherein the pairs of nuclei are active and selected from the group comprising ¹H, ¹³C, ¹⁵N or ³¹P.

8. A method according to any preceding claim wherein the liquid crystalline medium is selected from the group comprising:
(i) dimyristoyl phosphatidylcholine:dihexanoylphosphatidylcholine, in aqueous solution;
(ii) ditridecylphosphatidylcholine:dihexylphosphatidylcholine, in aqueous solution or;
(iii) an aqueous solution of cetylpyridinium chloride:hexanol in NaCl.

9. A method according to any preceding claim further comprising the step of isotopic enrichment with an NMR active stable isotope prior to generation of the high resolution NMR correlation spectra, wherein the ligand or a ligand library, the specific target molecule or both are enriched.

10. A method according to claim 9 wherein the enriching NMR active stable isotope is selected from the group consisting of: ¹³C, ¹⁵N, ³¹P or ²H, or a mixture of such isotopes or radioactive isotopes thereof in any combination, or any other NMR active stable isotope which occurs in the ligand.

11. A method according to any preceding claim wherein the target molecule is biochemically derivatised such that it is bound strongly to a chemical species that comprises a matrix of the liquid-crystalline medium, or possesses inherent capacity to do so.

12. A method according to claim 11 wherein the derivitisation comprises any one of the following procedures:
(i) myristoylation at one or more positions on the target molecule;
(ii) presence of a membrane spanning domain or glycosyl-phosphatidylinositol membrane anchor either inherently or by genetic engineering of an over-expressed protein or;
(iii) covalent attachment of the target molecule to functional groups on the liquid crystalline medium by chemical means.

13. Use of a method according to claim 1 for screening a library of ligands so as to select a candidate therapeutic comprising a ligand or ligands with appropriate biological activity.

14. Use according to claim 13 further including any one or more of the features recited in claims 2-12.

15. Use according to either claim 13 or 14 further comprising the step of mixing the selected ligand identified as a candiadate therapeutic, or derivative or homolgue thereof with a pharmaceutically acceptable carrier.

16. A method for the production of a pharmaceutical composition comprising identifying an agent ligand or ligands by the method as recited in any one of claims 1-15, and furthermore mixing the agent identified, or derivative or homologue thereof with a pharmaceutically acceptable carrier.
